(19) United States
(12) Patent Application Publication
(10) Pub. No.: US 2011/0171273 A1
(43) Pub. Date: Jul. 14, 2011

(54) STABLE ANTI-NAUSEA ORAL SPRAY FORMULATIONS AND METHODS

(76) Inventors: Frank E. Blondino, Easton, PA (US); Carrie Chen, East Brunswick, NJ (US)

(21) Appl. No.: 13/053,673

(22) Filed: Mar. 22, 2011

Related U.S. Application Data
(63) Continuation of application No. 12/004,409, filed on Dec. 21, 2007, now abandoned.
(60) Provisional application No. 60/876,484, filed on Dec. 22, 2006.

(51) Int. Cl. A61K 31/4178; A61K 9/00; A61P 1/08; B67D 7/58

(52) U.S. Cl. 424/400; 514/397; 222/372

(57) ABSTRACT

Stable formulations of selective 5-hydroxytryptamine receptor antagonists for oral spray administration for absorption by the oral mucosa and related methods of preparation and administration are provided. A preferred embodiment includes ondansetron in a concentration of about 5.1 to about 5.2% w/w; propylene glycol in a concentration of about 60.1 to about 60.3% w/w; water in a concentration of about 5.3 to about 5.4% w/w; and ethanol in a concentration of about 27.1 to about 27.3% w/w. Additional preferred embodiments are preservative free and/or non-aqueous or primarily non-aqueous.

This application claims priority to U.S. Provisional Patent Application No. 60/876,484, filed on Dec. 22, 2006, the disclosure of which is incorporated by reference herein in its entirety.

### FIELD OF THE INVENTION

The field of this invention is anti-nausea oral spray pharmaceutical formulations, methods of manufacturing such formulations, and their use in treating and preventing nausea and other conditions in human and non-human mammals.

### SUMMARY OF THE INVENTION

Preferred embodiments of the invention provide stable formulations of ondansetron hydrochloride and pharmaceutically acceptable salts thereof suitable for oral administration, and related methods of preparation and administration of ondansetron hydrochloride formulations. The invention provides stable, oral spray formulations in a simple, elegant format for fast onset of the active ingredient via absorption to the systemic circulatory system through the oral mucosa. In one embodiment, ondansetron hydrochloride is formulated in a non-aqueous or primarily non-aqueous, oral, propellant-free spray formulation at a concentration of about 0.1 to 7% w/w, more preferably 1 to 6% w/w, and most preferably about 5% w/w. Preferred, primarily non-aqueous, ondansetron hydrochloride formulations comprise, for example, (1) ondansetron hydrochloride (e.g., 0.1-7% w/w), acesulfame potassium salt (e.g., 0-0.5% w/w), propylene glycol (e.g., 30-70%), glycyrrhizic acid (e.g., 0-15%), bitter mask (e.g., 0-10% w/w), peppermint oil (e.g., 0-1% w/w), dehydrated ethanol (e.g., 15-50% w/w), and purified water (e.g., 0-10% w/w); or (2) ondansetron hydrochloride, acesulfame potassium salt, neotame (e.g., 0-1% w/w), propylene glycol, glycyrrhizic acid, bitter mask, peppermint oil, dehydrated ethanol, and purified water; or (3) ondansetron hydrochloride, sucralose (e.g., 0-2% w/w), propylene glycol, glycyrrhizic acid, bitter mask, peppermint oil, strawberry flavor (e.g. 0-1% w/w), purified water, and dehydrated ethanol.

In one embodiment, the ondansetron oral spray formulation contains propylene glycol, ethanol, and water. In this embodiment, ondansetron HCl is present at about 4-6%, preferably 4.5-5.5%, and most preferably 5.1-5.2% w/w; propylene glycol is present at about 55-65%, preferably 57-62%, and most preferably 60.1-60.3% w/w; ethanol is present at about 25-30%, more preferably 26-29%, and most preferably 27.1-27.3% w/w; and water is present at about 4-6%, preferably 4.5-5.8%, and most preferably 5.3-5.4% w/w.

In another embodiment of the invention, a pharmaceutically effective amount of ondansetron hydrochloride is delivered to the systemic circulatory system of a mammal via actuation of a spray pump adapted for administration of the formulations to the oral mucosal surfaces to spray a unit dose volume of about 10 to 500 μl of the formulation, wherein the spray preferably has a median particle size of about 30 um to 150 μm and an ovality ratio of less than abut 2.0. In yet another embodiment of the invention, sugar-free ondansetron hydrochloride spray formulations are provided. Further embodiments of the invention provide preservative-free, non-aqueous or primarily non-aqueous ondansetron hydrochloride formulations and methods for their preparation.

Additional features and advantages of the invention will be set forth in the description which follows and will be apparent from the description or may be learned by practice of the invention.

### BRIEF DESCRIPTION OF THE DRAWINGS

FIG. 1 illustrates the concentration of Impurity D in preferred formulations of the invention over time under two stability testing conditions (40° C./75% RH and 25° C./60% RH);

FIG. 2 is an HPLC chromatogram of a resolution solution of ondansetron (18.526) and Impurity A (15.985) at 306 nm depicting peaks for Impurities C (5.569) and D (6.886);

FIG. 3 is an HPLC chromatogram of a resolution solution of ondansetron and Impurities C and D at 328 nm depicting a peak for Impurity A;

FIG. 4 is an HPLC chromatogram of a sample solution of Sunett® Formulation C for the analysis of ondansetron at 306 nm;

FIG. 5 is an HPLC chromatogram of a sample solution of Sunett® Formulation C for the analysis of ondansetron at 328 nm; and

FIG. 6 depicts the residence time of an exemplary formulation in the oral cavity at various dosing volumes.

### DETAILED DESCRIPTION OF THE INVENTION

Reference will now be made in detail to the presently preferred embodiments of the invention, which, together with the following examples, serve to explain the principles of the invention. It is to be understood that the application of the teachings of the present invention to a specific problem or environment will be within the capabilities of one having ordinary skill in the art in light of the teachings contained herein. Illustrative embodiments of the products of the present invention and processes for their preparation and use appear in the following examples.

Preferred embodiments of the present invention provide stable, preservative-free pharmaceutical compositions which are primarily non-aqueous solutions comprising a therapeutically effective amount of ondansetron hydrochloride. In one embodiment, the preferred compositions do not resort to use of a preservative, but instead achieve inhibition of microbial growth by including an alcohol, preferably at least about 20% w/w ethanol, in the formulation.

Ondansetron, as the free base or hydrochloride salt, is indicated to prevent nausea and vomiting associated with initial and repeat courses of emetogenic cancer chemotherapy, including high-dose cisplatin, and to prevent postoperative nausea and/or vomiting.

Ondansetron is a selective 5-HT₃ receptor antagonist inhibiting the serotonin stimulation of the 5-HT₃ receptor, which initiates the vomiting reflex.

Ondansetron can be supplied and employed in formulations according to the invention as a hydrochloride salt and as a free base. The hydrochloride salt is used, for example, in the injectable solution (2 mg/mL), oral tablets (4, 8, and 24 mg), and oral solution (0.8 mg/mL). The free base is used, for example, in the orally disintegrating tablets (4 and 8 mg). Chemically, the hydrochloride salt is referred to as (±)1,2,3,9-tetrahydro-9-methyl-3-[(2-methyl-1H-imidazol-1-yl)methyl]-4H-carbazol-4-one, monohydrochloride, dihydrate. The empirical formula of the hydrochloride salt is C₁₈H₁₉N₃O.HCl.2H₂O; representing a molecular weight of 365.9. The free base is referred to as (±)1,2,3,9-tetrahydro-9-methyl-3-[(2-methyl-1H-imidazol-1-yl)methyl]-4H-carbazol-4-one. The empirical formula of the free base is C₁₈H₁₉N₃O representing a molecular weight of 293.4. Both the free base and HCl salt forms are white to off-white powders and sensitive to light. The term “ondansetron,” as used herein, refers to both the free base and all pharmaceutically acceptable salt forms unless otherwise noted.

The formulations according to the invention may also contain additional active pharmaceutical ingredients, such as, for example, including other serotonin antagonists (e.g., dolasetron (Anzemet®), granisetron (Kytril®), and palonosetron (Aloxi®), dopamine antagonists (e.g., chlorpromazine (Thorazine®), droperidol (Inapsine®), metoclopramide (Reglan®), prochlorperazine (Compazine®), promethazine (Phenergan®), trimethobenzamide (Tigan®)), anticholinergic agents such as scopolamine (Transderm Scop®), and antihistamines (e.g., buclizine (Bucladin-S®), cyclizine (Marezine®), dimenhydrinate (Dramamine®), diphenhydramine (Benadryl®), and meclizine (Antivert®)) including salts thereof. Other drugs suitable for combination therapy include droperidol, dexamethasone, methylprednisolone (Medrol®), and metoclopramide including salts thereof.

Under stability analyses, the storage stable compositions of the present invention show remarkable maintenance of the initial concentration of ondansetron hydrochloride and reduced levels of impurities. For example, preferred formulations of the invention maintain ondansetron content between a concentration of 3.9 mg/spray pump actuation and 4.2 mg/spray actuation over a 15 month period at 25° C. and 60% RH, while the average impurity concentration was less than 0.1% for the 15 month period.

As used herein, “storage stable” means liquid pharmaceutical formulations which include ondansetron as an active ingredient, and in which the concentration of the active ingredient is substantially maintained during storage stability testing, and degradation products and/or impurities which are typically observed in storage stability testing of such formulations are absent or significantly reduced during storage stability testing. In one embodiment, storage stability is determined at a temperature range from about 5° C. to about 80° C., about 20° C. to about 70° C., or about 25° C. to about 60° C. In another embodiment, storage stability is determined at a relative humidity (“RH”) range from about 30% RH to about 90% RH, about 50% RH to about 65% RH, or about 65% RH to about 75% RH. Preferred time intervals for measuring storage stability range, for example, from about 1 week to 5 years, from about 2 weeks to about 4 months, or at intervals of 2 weeks, 4 weeks, 8 weeks, 12 weeks, 16 weeks, 7 months, and 12 months.

As used herein, the term “primarily non-aqueous” refers to spray formulations which include ondansetron and are free or substantially-free of water. Primarily non-aqueous formulations may include a minimal quantity of aqueous solvent. For example, in preferred primarily non-aqueous formulations of the invention, water is present only to the degree necessary to dissolve acesulfame potassium salt. Other preferred formulations such as those which do not contain acesulfame potassium salt, for example the sucralose containing formulation, may be entirely free of water, i.e., non-aqueous.

Preferred formulations of the invention contain ethanol and/or propylene glycol. Without being bound by theory, it is believed that the inclusion of propylene glycol and ethanol inhibits microbial growth in the formulation and leads to increased stability of the formulation. Other alcohols such as benzyl alcohol, the parabens (for example, butylparaben, methylparaben), glycerol, propylene glycol, chlorobutanol, phenol, phenoxyethanol, and phenylethyl alcohol, at appropriate concentrations, may be used in place of ethanol for this purpose. Thus, in accordance with one embodiment of the invention, it is not necessary to include an antimicrobial component or agent to ensure safe storage without the proliferation of pathogenic molds, yeasts, or bacteria. Preferred formulations of the invention are primarily non-aqueous permitting inclusion of a higher concentration of the active ingredient (e.g., ondansetron). It is believed that the non-aqueous nature of the preferred formulations of the invention contribute to their self-preserving qualities.

In another embodiment of the invention, various antimicrobials which are suitable for use in foods and other ingestible substances can be used in the present invention. Examples include the parabens (butylparaben, methylparaben, and propylparaben), propyl-p-hydroxybenzoates, sodium benzoate, and sorbic acid including salts thereof. A preferred antimicrobial agent is benzoic acid or salts thereof, e.g., sodium benzoate.

Preferred embodiments of the invention are directed to buccal spray formulations for fast onset of the active ingredient via absorption to the systemic circulatory system through the oral mucosa. Therefore, preferred spray formulations of the invention maximize absorption to the systemic circulatory system and minimize or avoid absorption by other body systems (e.g., lungs, digestive system). The size of the spray particles contributes to whether the particles are absorbed into body systems other than the oral mucosa/circulatory system (e.g., lungs). For example, smaller sized particles are more likely to be inhaled. By “buccal” herein we mean of, or pertaining to, the mouth and oral cavity, including but not limited to the oral mucosal surfaces of the tongue, cheeks, gums and/or sublingual surfaces.

In one embodiment, the percentage of the particles (droplets) of the spray formulation (e.g., after actuation of a spray pump) having a diameter of less than ten microns is less than about 2%, more preferably less than about 1.5%. In another embodiment, the median diameter of the spray particles is from about 30 microns to about 150 microns, more preferably from about 60 microns to about 120 microns (e.g., Table 1).

The ellipticity or ovality ratio of the spray pattern indicates whether the spray is symmetrical. The ovality is defined as the ratio of Dₘₐₓ and Dₘᵢₙ. Dₘₐₓ is defined as the largest chord, in mm, that can be drawn within the spray pattern that crosses the COMw (i.e., center of mass of the spray pattern) in base units. Dₘᵢₙ is described as the smallest chord, in mm, that can be drawn within the spray pattern that crosses the COMw in base units. COMw is defined as the center of mass of the detected spray pattern, where each pixel's intensity is taken into account. The ovality ratio of the spray pattern indicates whether the spray is symmetrical. It is believed that the more symmetrical the oval shape of the pattern of spray particles, the more likely the particles will evenly cover the oral mucosa. In accordance with a preferred embodiment of the invention, the ovality ratio of the pattern is less than about 2.0, more preferably less than about 1.5 (Table 1). In another embodiment, increasing the viscosity of the formulation decreases the ovality of the spray pattern.

TABLE 1
Comparison of Standard Spray Pumps and High Viscosity Spray Pumps: Droplet size distribution and spray pattern

| | PG/H₂O | | | | | | PG/EtOH | | | | | |
|---|---|---|---|---|---|---|---|---|---|---|---|---|
| | High Viscosity | | | Standard | | | High Viscosity | | | Standard | | |
| | Spray Pumps | | | Spray Pumps | | | Spray Pumps | | | Spray Pumps | | |
| Vol. | 0.87 | 0.87 | 0.87 | 1.13 | 0.92 | 0.96 | 1.08 | 0.98 | 1.09 | 1.15 | 1.12 | 1.11 |
| <10.0 μm (%) | | | | | | | | | | | | |
| Dv (50.0) (μm) | 77.28 | 76.75 | 76.67 | 58.79 | 70.01 | 69.44 | 55.94 | 58.55 | 55.28 | 50.16 | 51.46 | 50.71 |
| Dv (90.0) (μm) | 131.56 | 132.42 | 134.25 | 114.36 | 127.05 | 124.74 | 114.57 | 114.34 | 110.94 | 109.58 | 108.97 | 112.59 |
| Ovality ratio | 1.240 | 1.242 | 1.240 | 1.289 | 1.356 | 1.316 | 1.525 | 1.482 | 1.530 | 1.354 | 1.387 | 1.375 |

In preparing the formulations of the present invention, the active ondansetron hydrochloride component may be incorporated into an aqueous solution. Preferably, ethanol and/or propylene glycol are used as solvents in the formulations of the invention. In one embodiment, water is optional and may be included, for example, in a minimal amount to serve as a solvent for taste masking components (e.g., acesulfame potassium salt, FCC). However, other solvents may be used which aid in solubilizing ondansetron hydrochloride and/or other components of the preferred spray formulations. These may include, for example, aliphatic alcohols, benzyl alcohol, glycerin, glycofurol, and polyethylene glycol.

The formulations can contain a propellant for delivery as an aerosol spray or can be propellant-free and delivered by a metered valve spray pump. Suitable propellants include, but are not limited to, hydrocarbons (butane, propane, etc.), chlorofluorocarbons (CFC-11, CFC-12, etc.), hydrofluorocarbons (HFA-134a, HFA-227ea, etc.), and ethers (dimethylether, diethylether, etc.).

In one embodiment of the invention, ondansetron hydrochloride formulations are provided which do not contain sweetening, taste masking, or flavoring agents. However, sweetening, taste masking, or flavoring agents such as Splenda® (sucralose), sorbitol, sucrose, neotame, bitter mask, peppermint oil, strawberry flavor, glycyrrhizic, or Sunett® (acesulfamate K) can be added if desired.

Various flavors or flavoring agents may be included to impart a pleasant taste. A pleasant taste is particularly important when the formulation is intended for administration to children or animals. Numerous flavors that are commonly used in pharmaceuticals, foods, candies and beverages are also suitable for use in the present invention. Examples include fruit, peppermint, licorice, bubble gum, and other flavors.

The formulations of the present invention can be prepared by various methods. One embodiment of a manufacturing method for Formula A is as follows. Preferably, Sunett® (e.g., acesulfame potassium salt, FCC) is dissolved in purified water, USP. This “Sunett® Solution” is then added later in the manufacturing process. Next, ondansetron HCl, USP is dissolved in propylene glycol, USP. It is preferred that the ondansetron is completely dissolved in propylene glycol, USP before adding any other excipients. Following dissolution, the ingredients are preferably added in the following order with constant stirring and thorough mixing between each addition: Magnasweet® (glycyrrhizic acid, FCC); Bitter Mask, water, Sunett® Solution, peppermint oil, NF, and dehydrated ethanol, USP. Preferably, dehydrated ethanol, USP is added last and after complete dissolution and mixing of previous ingredients. The final solution is preferably mixed well.

Formula A:

| Item# | | mg/g |
|---|---|---|
| 1 | Sunett ® (Acesulfame potassium salt, FCC) | 2.6 |
| 2 | Purified Water, USP | 48.6 |
| 3 | Ondansetron HCl, USP | 51.2 |
| 4 | Propylene Glycol, USP | 562.9 |
| 5 | MagnaSweet ®, FCC (Glycyrrhizic acid) | 51.2 |
| 6 | Bitter Mask | 25.6 |
| 7 | Peppermint Oil, NF | 5.1 |
| 8 | Dehydrated Ethanol, USP | 252.8 |

The solution can then be packaged into any suitable containers. Preferred containers are pharmaceutically acceptable glass, PET, and HDPE bottles with a capacity of between 1 and 100 mL. To ensure long-term photostability amber glass can be utilized. Additionally, if PET or HDPE is chosen, the bottle may be opaque to ensure long-term photostability.

The formulations are preferably dispensed using a metered pump device capable of delivering between 10 and 500 mcL. Pumps commonly used for dispensing nasal sprays are suitable for use with these formulations. In one embodiment, the pump and actuator may be modified such that the spray is dispensed horizontally to the bottle. This will allow easy dispensing to the mouth of the patient. The actuator may include an extension, if desired, to facilitate delivery to the buccal area of humans or animals.

The present invention also provides methods of treating various conditions in a subject (e.g., prevention of nausea and vomiting, chemotherapy-induced emesis, and post-operative nausea and vomiting). The methods include administering to a subject in need of treatment a storage stable pharmaceutical composition according to the invention. In one embodiment, the subject is a human; in another embodiment the subject is a non-human mammal, preferably selected from the group of dogs, cats, horses, cattle, sheep, and swine. The storage stable pharmaceutical composition can be administered to a patient in a dosage range of, for example, 0.1 mg to about 260 mg per day, preferably about 1 mg to about 64 mg per day, and more preferably 2 to 48 mg per day.

It is to be understood that application of the teachings of the present invention to a specific problem or environment will be within the capability of one having ordinary skill in the art in light of the teachings contained herein. The present invention is more fully illustrated by the following non-limiting examples.

### EXAMPLE 1

A physical stability study was conducted by placing 60/40 PG/H₂O and PG/EtOH solutions containing 5% API (e.g., ondansetron HCl) in a 5° C. refrigerator. The solution with a PG/H₂O solvent system crystallized after 2 days. The solution with a PG/EtOH solvent system remained in solution for more than 30 days.

Stability studies for formulations B and C (Tables 2 and 3) were conducted by preparing a 250 mL batch for each formulation, along with corresponding placebos. An aqueous solution of 20% Sunett® (w/v) was prepared and used to allow for more rapid mixing. To limit the total amount of water in the formulation at 5%, the amount of water added was reduced according to the amount of the Sunett® solution added.

TABLE 2
Sunett ® Formulation B

| Ingredient | w/v |
|---|---|
| Ondansetron HCl | 5.0% |
| Propylene Glycol | 55.0% |
| Purified Water | 2.5% |
| 20% Sunett ® Sol. | 2.5% |
| Magnasweet ® | 5.0% |
| Bitter Mask | 2.5% |
| Peppermint Oil | 0.5% |
| Ethanol | 28.0% |

TABLE 3
Sunett ® Formulation C

| Ingredient | w/v |
|---|---|
| Ondansetron HCl | 5.0% |
| Propylene Glycol | 55.0% |
| Purified Water | 3.75% |
| 20% Sunett ® Sol. | 1.25% |
| Magnasweet ® | 5.0% |
| Bitter Mask | 2.5% |
| Peppermint Oil | 0.5% |
| Ethanol | 28.0% |

Formulation B was preferred over formulation C due to the higher concentration of sweetener, which results in a better taste profile. Tables 4 and 5 provide stability data for Formulations B and C respectively.

TABLE 4
Stability data of Sunett ® Formulation B

| | | | Average Ond. | | Normalized | | | Average | |
|---|---|---|---|---|---|---|---|---|---|
| | | | HCl | Ondansetron | Ond. | | | Impurity D | |
| Time | Storage | Spray Wt. | Concentration | Content | Concentration | L.C./ | Normalized | Conc. | % Impurity |
| Point | Condition | (mg) | (μg/ml) | (mg/actuation) | (mg/100 μl) | actuation | L.C./100 μl | (μg/ml) | D |
| Initial | N/A | N/A (bulk) | 48.25 | N/A | 3.87 | N/A | 96.7% | 0.0010 | 0.002 |
| 2 weeks | 25° C./60% RH | 100.1 | 51.85 | 4.16 | 4.06 | 103.9% | 101.4% | 0.0023 | 0.004 |
| 4 weeks | 25° C./60% RH | 99.8 | 51.89 | 4.16 | 4.07 | 104.0% | 101.8% | 0.0028 | 0.006 |
| 8 weeks | 25° C./60% RH | 98.6 | 51.18 | 4.10 | 4.07 | 102.6% | 101.7% | 0.0022 | 0.004 |
| 12 weeks | 25° C./60% RH | 99.7 | 49.91 | 4.00 | 3.92 | 100.1% | 98.0% | 0.0037 | 0.009 |
| 16 weeks | 25° C./60% RH | 98.0 | 50.95 | 4.09 | 4.07 | 102.1% | 101.8% | 0.0058 | 0.011 |
| 7 months | 25° C./60% RH | 98.1 | 50.42 | 4.04 | 4.03 | 101.1% | 100.7% | 0.0086 | 0.017 |
| 2 weeks | 40° C./75% RH | 95.8 | 49.60 | 3.98 | 4.06 | 99.4% | 101.4% | 0.0082 | 0.017 |
| 4 weeks | 40° C./75% RH | 100.2 | 52.05 | 4.17 | 4.07 | 104.3% | 101.7% | 0.0162 | 0.031 |
| 8 weeks | 40° C./75% RH | 99.3 | 51.57 | 4.14 | 4.07 | 103.4% | 101.7% | 0.0249 | 0.048 |
| 12 weeks | 40° C./75% RH | 88.2 | 44.48 | 3.57 | 3.95 | 89.2% | 98.8% | 0.0346 | 0.085 |
| 16 weeks | 40° C./75% RH | 98.4 | 50.76 | 4.07 | 4.04 | 101.8% | 101.0% | 0.0488 | 0.096 |
| 7 months | 40° C./75% RH | 99.6 | 50.82 | 4.08 | 4.00 | 101.9% | 99.9% | 0.0910 | 0.179 |

Average Ond. HCl Concentration = Average of 3 data points from analyst printout
Calculation for Ondansetron content (mg/actuation) = Average Ond. HCl Concentration/365.86 * 293.37 * 0.1
Calculation for Normalized Ond. Concentration (mg/100 μl) = Ondansetron (mg/actuation)/(Spray wt./97.69)
Calculation for L.C./actuation = Ondansetron (mg/actuation)/4.00
Calculation for Normalized L.C./100 ul = Normalized Ond. Concentration (mg/100 μl)/4.00
Specifications:
Spray weight: 83.0 mg-112.3 mg; % L.C.: 85%-115%; Impurity D: no more than 0.10% (see Example 2)

TABLE 5
Stability data for Formulation C

| | | | Average Ond. | | Normalized | | | Average | |
|---|---|---|---|---|---|---|---|---|---|
| | | | HCl | Ondansetron | Ond. | | | Impurity | |
| | Storage | Spray Wt. | Concentration | Content | Concentration | L.C./ | Normalized | D Conc. | % Impurity |
| Time Point | Condition | (mg) | (μg/ml) | (mg/actuation) | (mg/100 μl) | actuation | L.C./100 μl | (μg/ml) | D |
| Initial | N/A | N/A (bulk) | 48.41 | N/A | 3.88 | N/A | 97.0% | 0.0021 | 0.004 |
| 2 weeks | 25° C./60% RH | 99.3 | 51.59 | 4.14 | 4.07 | 103.4% | 101.7% | 0.0022 | 0.004 |
| 4 weeks | 25° C./60% RH | 98.9 | 52.01 | 4.17 | 4.12 | 104.3% | 103.0% | 0.0028 | 0.005 |
| 8 weeks | 25° C./60% RH | 100.7 | 51.16 | 4.10 | 3.98 | 102.6% | 99.5% | 0.0027 | 0.005 |
| 12 weeks | 25° C./60% RH | 98.4 | 48.83 | 3.92 | 3.89 | 97.9% | 97.2% | 0.0039 | 0.008 |
| 16 weeks | 25° C./60% RH | 96.8 | 49.90 | 4.00 | 4.04 | 100.0% | 101.0% | 0.0056 | 0.011 |
| 7 months | 25° C./60% RH | 98.9 | 51.13 | 4.10 | 4.05 | 102.5% | 101.2% | 0.0086 | 0.017 |
| 2 weeks | 40° C./75% RH | 98.6 | 51.13 | 4.10 | 4.06 | 102.5% | 101.6% | 0.0084 | 0.017 |
| 4 weeks | 40° C./75% RH | 100.9 | 52.24 | 4.19 | 4.06 | 104.7% | 101.4% | 0.0154 | 0.030 |
| 8 weeks | 40° C./75% RH | 101.0 | 51.63 | 4.14 | 4.00 | 103.5% | 100.1% | 0.0243 | 0.047 |
| 12 weeks | 40° C./75% RH | 102.4 | 52.07 | 4.18 | 3.98 | 104.4% | 99.6% | 0.0412 | 0.079 |
| 16 weeks | 40° C./75% RH | 98.7 | 50.74 | 4.07 | 4.03 | 101.7% | 100.7% | 0.0477 | 0.094 |
| 7 months | 40° C./75% RH | 101.8 | 52.21 | 4.19 | 4.02 | 104.7% | 100.4% | 0.0930 | 0.178 |

Calculations same as in Table 4.

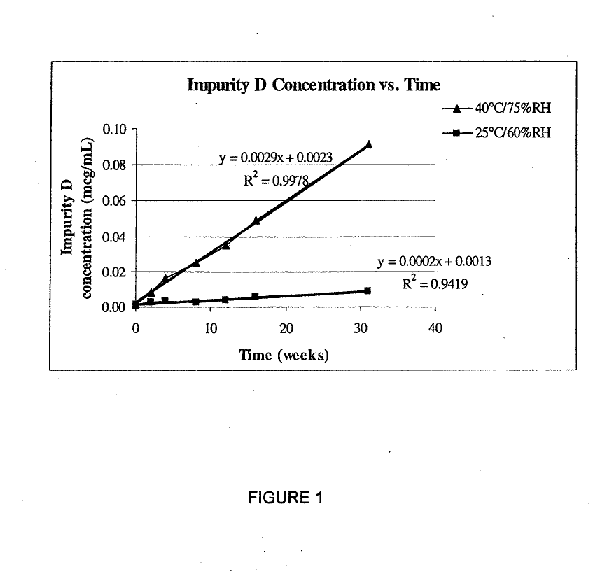

Ondansetron concentrations were normalized based on their spray weight. These normalized values may represent a more accurate representation of the stability of the formulations. Ondansetron concentration and label claim per actuation incorporates the variability of inconsistent spray weights, which can contribute to the wider data range fluctuation (see e.g., Table 4, 12 weeks 40° C./75% RH of formulation B). The Ondansetron concentration/actuation was 3.57 mg, which was 89.2% label claim, but the concentration/100 μl (the theoretical spray volume) was 3.95 mg, which comes out to be 98.8% label claim (FIG. 1).

The plot of concentration versus time for the formation of Impurity D indicates a zero order reaction. This is not what is typically observed in chemical degradation profiles. Typical profiles exhibit first order kinetics. However, Waterman and Adam have shown that under certain circumstances the degradation of a parent molecule, and thus the subsequent appearance of the degradation product, appear to exhibit zero order kinetics. K. C. Waterman and R. C. Adami “Accelerated aging: Prediction of chemical stability of pharmaceuticals”, Int J Pharm 23(1-2):101-125 (2005). The data indicate the current formulation is stable, i.e. Impurity D present at less than or equal to 0.1%, for 243 weeks at 25° C./60% RH. This exceeds the 104 weeks necessary for a 2 year shelf-life based upon the appearance of Impurity D.

### EXAMPLE 2

A cycling study was conducted. A set of bulk samples of Formulations B and C (Tables 4 and 5, respectively) and their placebos were stored in clear scintillation vials, wrapped in aluminum foil to protect them from light, and cycled between 5° C. in a refrigerator and the 40° C./75% RH stability chamber daily. After 29 days, a small white particle was observed in one of the three formulation B samples, and a small amount of white precipitation was observed in all of the formulation C samples, including the placebos. This observation was made immediately after 16 hours of refrigeration. After 8 hours of warming in the 40° C./75% RH stability chamber, the precipitate was still present. The presence of the precipitate in the placebo suggested that it was related to the excipient rather than the API.

To further investigate the cause of precipitation, six formulations were prepared and cycled between 5° C. refrigerator and 40° C./75% RH daily. The formulations were:

TABLE 6
Investigation of cause of precipitation

| | Sunett ® | Description |
|---|---|---|
| 1 | 0.5% | All ingredients |
| 2 | 0.0% | All ingredients |
| 3 | 0.5% | No Magnasweet |
| 4 | 0.5% | No Peppermint Oil |
| 5 | 0.5% | No Peppermint Oil, no Magnasweet |
| 6 | 0.5% | No Peppermint Oil, no Magnasweet, no Bitter Mask |

Formulations 5 and 6 from the above table precipitated out after one night of refrigeration. Formulation 3 precipitated out after two days. Formulations 1, 2, and 4 remained in solution for 35 days of daily cycling. The remaining solutions all contained Magnasweet®. It was concluded that a component of Magnasweet®, possibly the glycerol vehicle, was likely responsible for maintaining the solution system.

Another attempt to prevent precipitation was the addition of 0.9% benzyl alcohol. A comparative study of Formulation B with 5% water, with and without benzyl alcohol was conducted by preparing triplicate samples of both formulations and cycling them daily. All samples were free of precipitation at 40 days, after which the samples were left in the 5° C. refrigerator. They remained in solution for more than 6 months.

### EXAMPLE 3

To further investigate formulations with reduced precipitation, two alternative systems were investigated (Tables 7 and 8). The alternative neotame/Sunett® Formulation contains approximately the same amount of water as Sunett® Formulations B and C above. However, the amount of Sunett® is reduced. The alternative Splenda® Formulation does not contain any added water. Both formulations were stored for significant periods of time without any precipitation. Physical observations and chemical analysis were performed on the neotame/Sunett® and Splenda® formulations after 11 and 15 months, respectively (Tables 9 and 10). After 11 and 15 months of refrigeration, neither Formulation exhibited precipitation. Thus, Sunett® may have some effect on the physical stability of the product. In addition, removing Sunett® and water completely, as in the Splenda® Formulation, resulted in similar acceptable physical stability. These exemplary formulations would be suitably stable after 2 years of storage at room temperature

TABLE 7
Alternative Neotame/Sunett ® Formulation

| Ingredient | % w/w |
|---|---|
| Ondansetron HCl | 5.1 |
| Propylene glycol | 56.1 |
| MagnaSweet ® | 5.1 |
| Bitter Mask | 2.6 |
| Purified Water | 4.6 |
| 20% Sunett ® in water | 0.5 |
| Peppermint Oil | 0.5 |
| 0.5% Neotame in ethanol | 1.6 |
| Ethanol | 24.0 |

TABLE 8
Alternative Splenda ® Formulation

| Ingredient | % w/w |
|---|---|
| Ondansetron HCl | 5.1 |
| Propylene glycol | 57.3 |
| MagnaSweet ® | 5.2 |
| Bitter Mask | 2.6 |
| Peppermint Oil | 0.5 |
| Splenda ® | 0.2 |
| Ethanol | 29.2 |

TABLE 9
Stability Data For Alternative Neotame/Sunett ® Formulation

| | | | | Spray | | |
|---|---|---|---|---|---|---|
| Time | | Temp. | | Weight | % L.C. | % |
| Point | | □ C. | % R.H. | (mg) | Ondansetron | Imp. D |
| Initial | | N/A | N/A | N/A | 100.3 | 0.012 |
| Initial | | N/A | N/A | 97.5 | 100.6 | 0.012 |
| 3 | Days | 60 | N/A | 76.9 | 80.7 | 0.112 |
| 3 | Months | 25.0 | 60 | 97.9 | 100.8 | 0.014 |
| 3 | Months | 40.0 | 75 | 99.7 | 102.0 | 0.068 |
| 11 | Months | 25.0 | 60 | N/A | 98.2 | 0.033 |
| 11 | Months | 30.0 | 65 | N/A | 99.3 | 0.062 |
| 11 | Months | 40.0 | 75 | N/A | 97.9 | 0.292 |

TABLE 10
Stability Data for Alternative Splenda ® Formulation

| | | | | Spray | | |
|---|---|---|---|---|---|---|
| Time | | Temp. | | Weight | % L.C. | % |
| Point | | □ C. | % R.H. | (mg) | Ondansetron | Imp. D |
| Initial | | N/A | N/A | N/A | 96.6 | 0.011 |
| Initial | | N/A | N/A | 97.6 | 98.1 | 0.011 |
| 3 | Days | 60 | N/A | 95.5 | 97.2 | 0.118 |
| 2 | weeks | 25 | 60 | 97.0 | 99.1 | 0.011 |
| 2 | weeks | 40 | 75 | 97.9 | 99.3 | 0.028 |
| 4 | weeks | 25 | 60 | 97.3 | 99.3 | 0.013 |
| 4 | weeks | 40 | 75 | 99.2 | 101.0 | 0.044 |
| 15 | Months | 25 | 60 | N/A | 95.7 | 0.050 |
| 15 | Months | 30 | 65 | N/A | 95.1 | 0.105 |
| 15 | Months | 40 | 75 | N/A | 93.5 | 0.491 |

### EXAMPLE 4

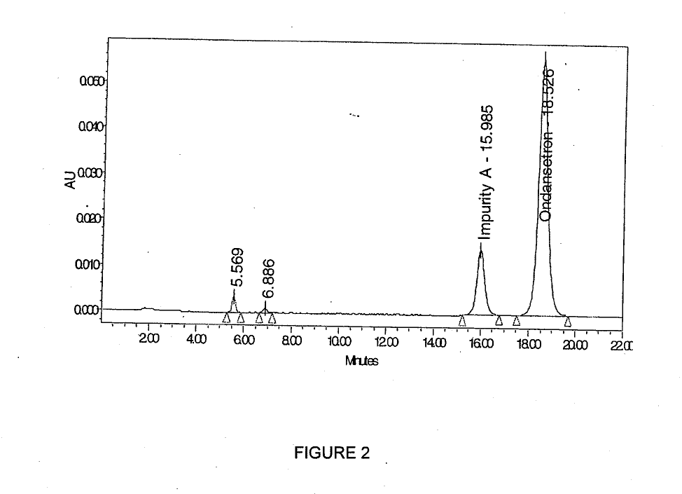

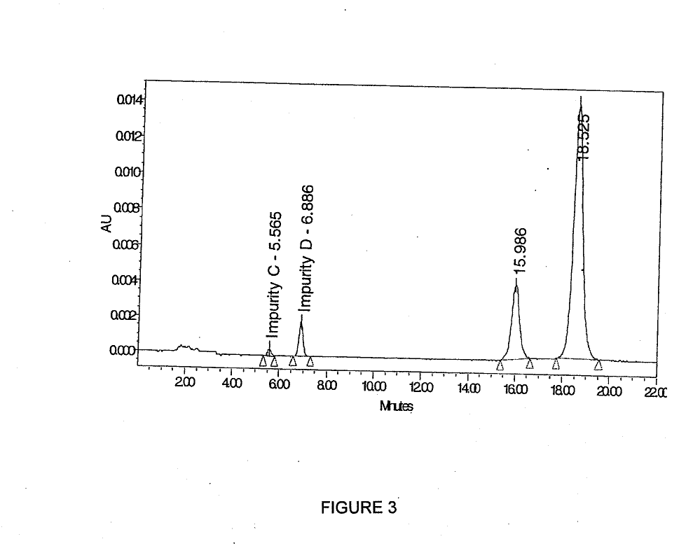

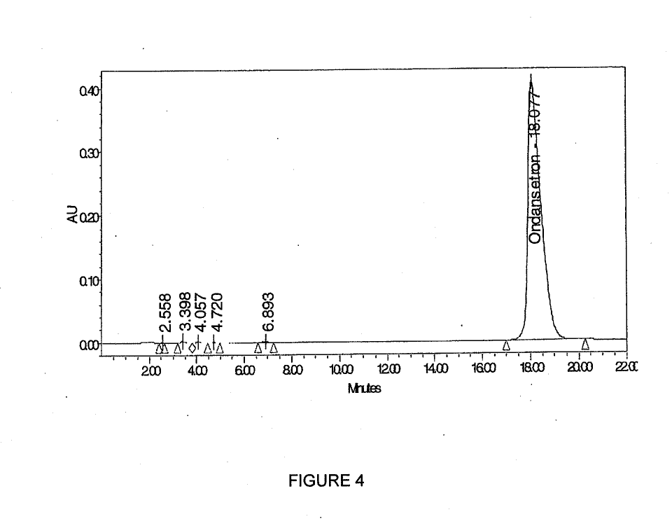

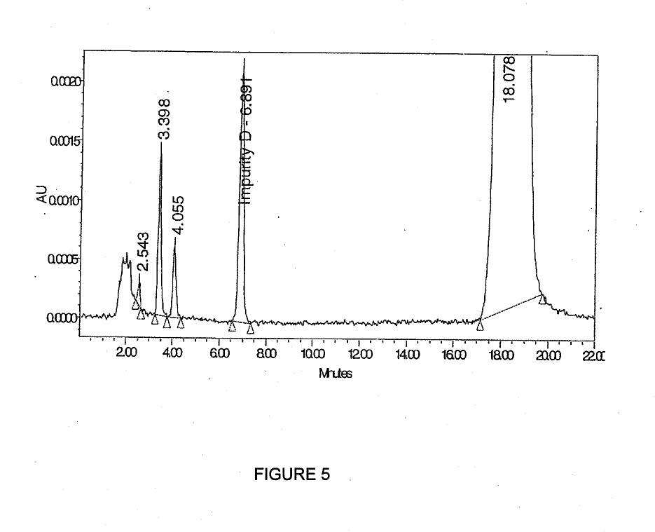

HPLC analysis revealed the relative retention time and relative response factor for five impurities and ondansetron as shown below. FIGS. 2-5 are an HPLC chromatogram of a resolution solution of Ondansetron and Impurity A at 306 nm depicting peaks for Impurities C and D; an HPLC chromatogram of a resolution solution of Ondansetron and Impurities C and D at 328 nm depicting a peak for Impurity A; an HPLC chromatogram of a sample solution of Sunett® Formulation C for the analysis of Ondansetron at 306 nm; and, an HPLC chromatogram of a sample solution of Sunett® Formulation C for the analysis of Ondansetron at 328 nm.

TABLE 11
Relative retention time and response factor of impurities

| Impurity | Relative Retention Time | Relative Response Factor |
|---|---|---|
| Impurity C | 0.36 | 1.64 |
| Impurity D | 0.43 | N/A |
| Impurity G | 0.74 | N/A |
| Impurity A | 0.86 | 1.19 |
| Ondansetron | 1.00 | 1.00 |

Note:
Except Impurity D, all other impurities are calculated based on the sample solutions prepared for ondansetron.

### EXAMPLE 5

Stability studies were conducted for formulations utilizing saccharin sodium to determine the long term compatibilities of saccharin sodium and bitter mask with the drug solution.

The exemplary formulations use 55% propylene glycol (“PG”). Saccharin Sodium was prepared as a 2% aqueous solution for ease of solubilization. The percentage of water in the primary solvent system was 60%140% PG/H₂O or PG/Ethanol (“EtOH”).

TABLE 12
Saccharin Sodium Formulation 2

| Ingredient | w/v |
|---|---|
| Ondansetron HCl | 5.0% |
| Propylene Glycol | 55.0% |
| 2% Saccharin Sodium Solution | 11.0% |
| Bitter Mask | 3.0% |
| Strawberry Flavor | 3.5% |
| Ethanol | ~21% |

TABLE 13
Saccharin Sodium Formulation 3

| Ingredient | w/v |
|---|---|
| Ondansetron HCl | 5.0% |
| Propylene Glycol | 55.0% |
| 2% Saccharin Sodium Solution | 13.0% |
| Bitter Mask | 4.0% |
| Strawberry Flavor | 4.5% |
| Water | ~21% |

TABLE 14
Saccharin Sodium Formulation 4

| Ingredient | w/v |
|---|---|
| Ondansetron HCl | 5.0% |
| Propylene Glycol | 55.0% |
| 2% Saccharin Sodium Solution | 11.0% |
| Bitter Mask | 3.0% |
| Strawberry Flavor | 3.5% |
| Oleic Acid | 2.0% |
| Ethanol | ~20% |

TABLE 15
Saccharin Sodium Formulation 5

| Ingredient | w/v |
|---|---|
| Ondansetron HCl | 5.0% |
| Propylene Glycol | 55.0% |
| 2% Saccharin Sodium Solution | 13.0% |
| Bitter Mask | 4.0% |
| Strawberry Flavor | 4.5% |
| Benzalkonium Chloride | 0.1% |
| Water | ~21% |

Formulations 2 and 4 were filled to volume with ethanol (EtOH), with formulation 4 containing an extra 2% of oleic acid and formulations 3 and 5 were filled to volume with H₂O, with formulation 5 containing an extra 0.1% of benzalkonium chloride. During preparation of formulation 4 the oleic acid was not soluble with the rest of the solution, and this formulation was eliminated as a candidate. Formulations 2, 3, and 5 were placed in three different storage conditions: 5° C. refrigerator; 25° C./60% RH; and 40° C./75% RH stability chambers. After one day, all three formulations were placed in the refrigerator and the 25° C./60% RH stability chamber. Formulation 2 showed very little precipitation compared to Formulations 3 and 5 (Tables 12). All three formulations stored in the 40° C./75% RH stability chamber remained in solution. However, after 45 days, formulation 2 stored in the 40° C./75% RH stability chamber did not show significant precipitation. The formulations had an average spray content of 97.3% label claim and 0.02% impurity D at day 45 in the 40° C./75% RH stability chamber.

The cause of precipitation of ondansetron in formulations 2, 3, and 5 in the refrigerator and in room temperature was investigated to evaluate the effect of saccharin: Formulation 2 was prepared 3 ways: without API, without saccharin solution (replaced by water), and without both API and saccharin. These samples remained in solution after incubation in the refrigerator overnight. Next, the samples were scratched and cycled between the refrigerator and the 40° C./75% RH stability chamber daily for 3 days and subsequently stored in the refrigerator for 3 months. The solutions remained clear. It is believed that the precipitation of ondansetron was caused by the coexistence of ondansetron HCl and saccharin in one solution. The study was continued by preparing formulations 2 and 3 based on the original formulations and using the same formulations where the saccharin solution was replaced with water. These four formulations were prepared in triplicate. After one night of refrigeration, the ondansetron in the samples containing saccharin sodium precipitated out. The ondansetron in two out of three samples of formulation 3 without saccharin sodium precipitated out as well. However, the ondansetron in the formulation 2 without saccharin sodium stayed in solution for 3 months.

TABLE 16
Effect of saccharin solution and water on formulations 2 and 3

| | Formulation 2 | | | Formulation 3 | | |
|---|---|---|---|---|---|---|
| | (QS'd with EtOH) | | | (QS'd with H₂O) | | |
| | Bottle 1 | Bottle 2 | Bottle 3 | Bottle 1 | Bottle 2 | Bottle 3 |
| With | Precipitated | Precipitated | Precipitated | Precipitated | Precipitated | Precipitated |
| Saccharin | | | | | | |
| solution | | | | | | |
| Saccharin | In solution | In solution | In solution | Precipitated | Precipitated | In solution |
| solution | | | | | | |
| replaced | | | | | | |
| by H₂O | | | | | | |

To investigate the cause of precipitation in formulation 3 without saccharin, three additional samples were made which included 5%, 10%, and 15% EtOH. Following incubation in the 5° C. refrigerator, the samples were analyzed. The ondansetron in the sample containing 5% EtOH stayed in solution for more than a week, the ondansetron in the sample containing 10% EtOH precipitated out in one day, and the ondansetron in the sample containing 15% EtOH stayed in solution for more than a day and less than a week. This indicated that an optimal EtOH concentration was preferred to maintain a homogeneous solution when using this combination of excipients. Three samples of formulation 5 were made by replacing the 2% saccharin sodium solution with water and replacing 17% water (QS part) with EtOH; and adding only 0.1%, 0.15%, and 0.2% saccharin sodium to each sample. The ondansetron in these three formulations precipitated overnight as well. It was suspected that water contributed to the instability. Three samples of formulation 2 were made without saccharin and without water, along with another three samples made without saccharin and with only 5% water. These six samples were placed in the 5° C. refrigerator. The ondansetron in one out of three samples with 5% water precipitated after 2½ weeks, and the ondansetron in the other two samples precipitated after 6 weeks. The ondansetron in the 3 samples with no water remained in solution for 12 weeks. These experiments led to the conclusion that saccharin sodium was not soluble in the formulation solution, and large amounts of water will cause physical instability of the formulation as well.

### EXAMPLE 6

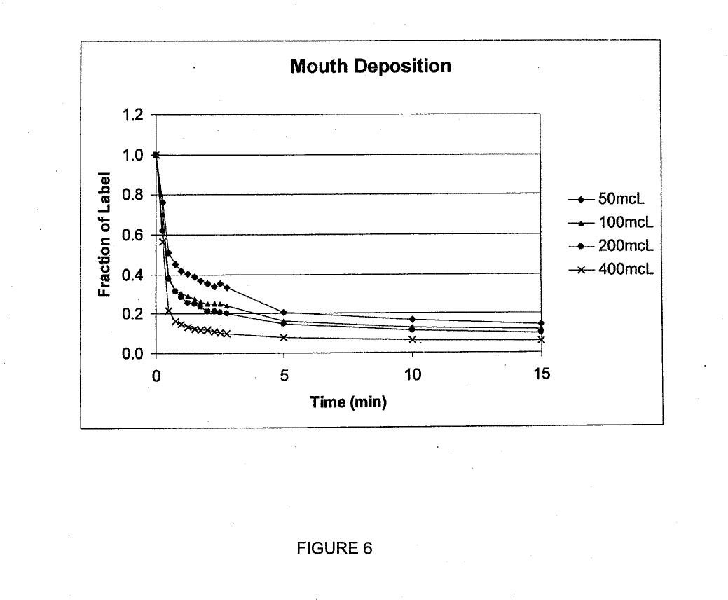

Studies were conducted to determine the residence time in the oral cavity. Briefly, a placebo formulation was radiolabeled with Technetium 99m-DTPA. Various volumes of the radiolabeled formulation were delivered to 8 human subjects after receiving necessary IRB clearance and consent. After the defined volume was delivered, deposition was imaged using gamma scintigraphy through the entire gastrointestinal tract (oral cavity through large intestine). Oral cavity imaging was conducted for the first 3 minutes at 15 second intervals, followed by imaging at 5, 10, 15, 20, 30, 45, 60, 90, 120, 150, 180, 210, and 240 minutes post dosing. After the 3 minute interval, lower gastrointestinal tract images were taken at the time points above. From this data, it was determined that it was beneficial to minimize the dosing volume (FIG. 6). From FIG. 6, it was observed that the lowest dosing volume, 50 mcL, resulted in the longest residence time of the label in the mouth. At 5 minutes, there was still approximately 20% of the label still present in the oral cavity. There is approximately half as much residing in the oral cavity at the same time point for 400 mcL. In addition, there was little difference between 100 mcL and 200 mcL, although the percent remaining in the mouth at 5 minutes was slightly less than that of the 50 mcL dose but more than that observed after a 400 mcL dose. Longer residence times in the oral cavity will increase oral mucosal absorption. This longer residence time effectively increases the exposure of the oral mucosa to the compound and increases transport across the mucosal membrane.

### EXAMPLE 7

As described above, it is advantageous to give embodiments a pleasant taste for administration to children or non-human animals. An embodiment including a flavoring ingredient is formulated as shown in Tables 17 and 18. The ranges given in Tables 17 and 18 are illustrative of one embodiment using MagnaSweet and Sucralose in combination with one or more flavoring ingredients.

TABLE 17
Flavored Sucralose Formulation

| Ingredient | w/w |
|---|---|
| Ondansetron HCl | 5.13-5.14% |
| MagnaSweet | 1.03% |
| Propylene Glycol | 60.16-60.30% |
| Sucralose | 0.77% |
| Flavoring ingredient(s) | 0.15-0.36% |
| H₂O | 5.36-5.37% |
| EtOH | 27.19-27.25% |

TABLE 18
Flavored Sucralose Formulation

| Ingredient | w/w |
|---|---|
| Ondansetron HCl | 5.1-5.2% |
| MagnaSweet | 1.0-1.1% |
| Propylene Glycol | 60.1-60.3% |
| Sucralose | 0.5-0.8% |
| Flavoring ingredient(s) | 0.1-0.4% |
| H₂O | 5.3-5.4% |
| EtOH | 27.1-27.3% |

Flavors for use in these exemplary formulations include strawberry, mint, fruit punch, strawberry banana, and combinations thereof.

The above description and examples are only illustrative of preferred embodiments which achieve the objects, features, and advantages of the present invention, and it is not intended that the present invention be limited thereto.

What is claimed as new and desired to be protected by Letters Patent of the United States is:

1. A pharmaceutical oral spray product comprising a selective 5-hydroxytryptamine receptor antagonist formulation in a spray pump container, wherein the formulation is primarily non-aqueous and when a unit dosage volume of about 10 to about 500 μL of the formulation is sprayed, the spray has a median particle diameter of about 30 μm to about 150 μm and an ovality ratio of less than about 2.0.

2. The oral spray of claim 1, wherein the spray has a median particle diameter of about 60 μm to about 120 μm.

3. The oral spray of claim 1, wherein the spray has an ovality ratio of less than about 1.5.

4. The oral spray of claim 1, wherein the formulation further comprises a flavoring ingredient.

5. The oral spray of claim 4, wherein the flavoring ingredient is sucralose.

6. The oral spray of claim 4, wherein the flavoring ingredient is selected from the group consisting of peppermint oil, strawberry flavor, neotame, bitter mask, glycyrrhizic, acesulfamate potassium, sucrose, and sorbitol.

7. The oral spray of claim 1, wherein the formulation further comprises a propellant.

8. The oral spray of claim 7, wherein the propellant is selected from the group consisting of hydrocarbons, chlorofluorocarbons, hydrofluorocarbons, and ethers.

9. The oral spray of claim 1, wherein the formulation further comprises a solvent.

10. The oral spray of claim 9, wherein the solvent is an alcohol.

11. The oral spray of claim 9, wherein the solvent is selected from the group consisting of H₂O, ethanol, propylene glycol.

12. The oral spray of claim 1, wherein the formulation is storage stable.

13. The oral spray of claim 1, wherein the selective 5-hydroxytryptamine receptor antagonist is ondansetron.

14. The oral spray of claim 13, wherein ondansetron is present in about 0.1 to about 7% w/w.

15. The oral spray of claim 14, wherein ondansetron is present in about 5.0 to about 5.2% w/v.

16. The oral spray of claim 13, wherein the formulation comprises about 15 to about 50% w/w ethanol.

17. The oral spray of claim 16, wherein the ethanol is present in about 20 to about 29.2% w/v.

18. The oral spray of claim 1, wherein the formulation is non-aqueous.

19. The oral spray of claim 1, wherein the formulation is preservative-free.

20. The oral spray of claim 1, wherein the formulation is non-aqueous and preservative free.

21. An oral spray composition, comprising:
ondansetron in a concentration of about 4 to about 6% w/w;
propylene glycol in a concentration of about 55 to about 65% w/w;
water in a concentration of about 4 to about 6% w/w; and
ethanol in a concentration of about 25 to about 30% w/w.

22. The oral spray of claim 21, comprising ondansetron in a concentration of about 4.5 to about 5.5% w/w;
propylene glycol in a concentration of abut 57 to about 62% w/w;
water in a concentration of about 4.5 to about 5.8% w/w; and
ethanol in a concentration of about 26 to about 29%.

23. The oral spray of claim 21, comprising ondansetron in a concentration of about 5.1 to about 5.2% w/w;
propylene glycol in a concentration of about 60.1 to about 60.3% w/w;
water in a concentration of about 5.3 to about 5.4% w/w; and
ethanol in a concentration of about 27.1 to about 27.3% w/w.

24. The oral spray of claim 21, further comprising one or more of a sweetening agent, a taste masking agent or a flavoring agent.

25. A method of treating a condition in a human or non-human animal comprising spraying a unit dose volume of about 10 to about 500 μL of a pharmaceutical composition on the oral mucosa of the animal, wherein the composition is primarily non-aqueous and the spray has a median particle size diameter of about 30 to 150 μm and an ovality ratio of less than about 2.0, wherein the composition comprises ondansetron and a solvent, and the ondansetron is absorbed through the oral mucosa to alleviate said condition.

26. The method of claim 25, wherein ondansetron is present in the composition at about 0.1 to about 7% w/w.

27. The method of claim 25, wherein ondansetron is present in the composition at about 5.0 to about 5.2% w/w.

28. The method of claim 25, wherein the ondansetron is administered in a dose from about 0.1 mg to about 260 mg per day.

29. The method of claim 28, wherein the ondansetron is administered in a dose from about 1 mg to about 64 mg per day.

30. The method of claim 29, wherein the ondansetron is administered in a dose from about 2 mg to about 48 mg per day.

31. The method of claim 25, wherein the solvent is selected from the group consisting of ethanol, water, propylene glycol, benzyl alcohol, aliphatic alcohol, glycerin, glycofurol, and polyethylene glycol.

32. The method of claim 25, wherein the condition is selected from the group consisting of nausea, vomiting, and emesis.
